(19) United States
(12) Patent Application Publication
(10) Pub. No.: US 2017/0325503 A1
(43) Pub. Date: Nov. 16, 2017

(54) ELECTRONIC CIGARETTE

(71) Applicant: HUIZHOU KIMREE TECHNOLOGY CO., LTD., Huizhou, Guangdong (CN)

(72) Inventor: Qiuming Liu, Shenzhen, Guangdong (CN)

(21) Appl. No.: 15/522,781

(22) PCT Filed: Oct. 28, 2014
(86) PCT No.: PCT/CN2014/089656

§ 371 Date: Apr. 28, 2017

(51) Int. Cl. A24F 47/00; A61M 11/04; H01M 10/04; H02J 7/00

(52) U.S. Cl. CPC A24F 47/008; A61M 11/044; H01M 10/0422; H02J 7/0045; H01M 10/0436

(57) ABSTRACT

An electronic cigarette, which comprises an atomization assembly and a battery assembly, the battery assembly is electrically connected to the atomization assembly. The atomization assembly comprises a light permeable e-liquid storage sleeve, an e-liquid storage cavity is formed in the e-liquid storage sleeve, an end of the e-liquid storage sleeve is an open end in communication with the e-liquid storage cavity, a connector is provided at the open end, an atomization core detachably connected to the connector is inserted in the connector. A battery sleeve of the electronic cigarette is directly detachably connected onto the e-liquid storage sleeve, which and prevents the shortcoming in which the atomization core is unplugged by the user when rotating the battery sleeve.

### FIELD OF THE INVENTION

The present invention related to a technical field of an electronic cigarette, and more particularly, to an electronic cigarette.

### BACKGROUND OF THE INVENTION

In the prior art, electronic cigarette comprises an atomization assembly and a battery assembly, the atomization assembly comprises an e-liquid storage sleeve and an atomization core which are connected to each other, the atomization core is detachably connected to the e-liquid storage sleeve, while the battery assembly is detachably connected to the atomization core, when the user disassembles the battery assembly from an end of the atomization core, if the connection strength between the atomization core and the e-liquid storage sleeve is smaller than the connection strength between the atomization core and the battery assembly, the user easily rotates the atomization core out from the e-liquid storage sleeve, while the atomization core and the battery assembly are still in connection, which causes a leakage of e-liquid and affects an experience of the user.

### SUMMARY OF THE INVENTION

The object of the present invention is to provide an electronic cigarette, aiming at the above drawback.

The technical solutions of the present application for solving the technical problems are as follows:

The present invention provides an electronic cigarette, comprising an atomization assembly and a battery assembly, the battery assembly is electrically connected to the atomization assembly and is configured for supplying electrical power to the atomization assembly, wherein the atomization assembly comprises a light permeable e-liquid storage sleeve, an e-liquid storage cavity configured for storing e-liquid is formed in the e-liquid storage sleeve, an end of the e-liquid storage sleeve is an open end in communication with the e-liquid storage cavity, a connector is provided at the open end, an atomization core detachably connected to the connector and configured for atomizing the e-liquid is inserted in the connector;

The battery assembly comprises a battery sleeve, a connecting structure configured for detachably connecting the battery sleeve with the e-liquid storage sleeve is provided at a connection of the battery sleeve and the e-liquid storage sleeve, a first electric connection assembly electrically connected to the atomization core is accommodated in an end of the battery sleeve;

the atomization core comprises an atomization sleeve, a second electric connection assembly electrically connected to the first electric connection assembly is provided at an end of the atomization sleeve, the end of the atomization sleeve (31) extends outside the e-liquid storage sleeve, the second electric connection assembly is inserted into the battery sleeve and is abutted against the first electric connection assembly, an atomization cavity is formed in the atomization sleeve, a heating wire assembly electrically connected to the second electric connection assembly is accommodated in the atomization cavity.

In the electronic cigarette provided in the present invention, the connecting structure is a threaded connecting structure or a bayonet connecting structure.

In the electronic cigarette provided in the present invention, the connecting structure comprises an external thread defined on an outer wall of the connector or the e-liquid storage sleeve, and an internal thread matched with the external thread and defined on the battery sleeve.

In the electronic cigarette provided in the present invention, the connecting structure comprises at least one buckle bulging on an outer wall of the e-liquid storage sleeve, and at least a clamping groove defined on an inner wall of the battery sleeve, the clamping groove is shaped in “L” and matched with the buckle.

In the electronic cigarette provided in the present invention, the clamping groove comprises an insertion channel and a rotation channel, the insertion channel and the rotation channel are smoothly connected to each other; the insertion channel is defined on an end of the battery sleeve and extends along an axis of the battery sleeve to a side opposite to the buckle, the rotation channel is connected to an end of the insertion channel opposite to the buckle and extends along a circumference of the battery sleeve.

In the electronic cigarette provided in the present invention, at least one inlet hole communicating with the atomization cavity and configured for inletting air and discharging the e-liquid in the atomization cavity is defined on an outer wall of the battery sleeve and the atomization sleeve, the inlet hole comprises a first inlet hole defined on the battery sleeve and a second inlet hole defined on the atomization sleeve.

In the electronic cigarette provided in the present invention, the second inlet hole is located at a wall of an end of the atomization cavity and is configured for discharging the e-liquid at the wall of atomization cavity, the end of the atomization cavity is close to the battery assembly.

In the electronic cigarette provided in the present invention, the first electric connection assembly is fixed on an inner wall of the battery sleeve, a plug-in groove plug-in connected to the second electric connection assembly is defined on a side of the first electric connection assembly towards the atomization core.

In the electronic cigarette provided in the present invention, the first electric connection assembly comprises a first internal electrode, a first insulation member and a first external electrode which are successively sheathed and connected from inside to outside, the plug-in groove is defined on the first external electrode.

In the electronic cigarette provided in the present invention, two installation grooves separated with each other and in communication with the e-liquid storage cavity are defined relatively and axially on an end of a sidewall of the atomization sleeve, the end of a sidewall of the atomization sleeve is opposite to the battery assembly, the heating wire assembly is built in the two installation grooves.

In the electronic cigarette provided in the present invention, the heating wire assembly comprises an e-liquid guide member configured for absorbing the e-liquid in the e-liquid storage cavity, and a heating wire configured for atomizing the e-liquid on the e-liquid guide member, two ends of the heating wire are respectively and electrically connected to the second electric connection assembly and realize an electrical connection with the battery assembly through the second electric connection assembly.

In the electronic cigarette provided in the present invention, a suction nozzle is connected to an end of the e-liquid storage sleeve, the end of the e-liquid storage sleeve is opposite to the atomization core, a vent pipe is sheathed in the e-liquid storage sleeve, two ends of the vent pipe are respectively inserted into the suction nozzle and the atomization core.

In the electronic cigarette provided in the present invention, an external end surface of the suction nozzle is a spherical surface.

In the electronic cigarette provided in the present invention, the atomization core further comprises an atomization cover sheathed on the atomization sleeve and the vent pipe and abutted against the heating wire assembly, an end of the vent pipe runs through the atomization cover and extends into the atomization cavity.

In the electronic cigarette provided in the present invention, a limiting convex part abutting against an end surface of the atomization cover is defined on an outside surface of the atomization sleeve.

In the electronic cigarette provided in the present invention, a convex configured for supporting the heating wire assembly is defined on an outside surface of the atomization sleeve.

Present invention further provides an electronic cigarette, comprising an atomization assembly and a battery assembly, the battery assembly is electrically connected to the atomization assembly and is configured for supplying electrical power to the atomization assembly, wherein the atomization assembly comprises a light permeable e-liquid storage sleeve, an e-liquid storage cavity configured for storing e-liquid is formed in the e-liquid storage sleeve, an end of the e-liquid storage sleeve is an open end in communication with the e-liquid storage cavity, a connector is provided at the open end, an atomization core detachably connected to the connector and configured for atomizing the e-liquid is inserted in the connector;

the battery assembly comprises a battery sleeve, the battery sleeve is detachably connected to the e-liquid storage sleeve through a threaded connecting structure or a bayonet connecting structure, a first electric connection assembly electrically connected to the atomization core is accommodated in an end of the battery sleeve;

the atomization core comprises an atomization sleeve, a second electric connection assembly electrically connected to the first electric connection assembly is provided at an end of the atomization sleeve, the end of the atomization sleeve extends outside the e-liquid storage sleeve, the second electric connection assembly is inserted into the battery sleeve and is abutted against the first electric connection assembly, an atomization cavity is formed in the atomization sleeve, a heating wire assembly electrically connected to the second electric connection assembly is accommodated in the atomization cavity.

To sum up, the implementation of the electronic cigarette of present invention has following beneficial effects: firstly, the battery sleeve is directly detachably connected to the e-liquid storage sleeve, which prevents an user from applying a force directly to the atomization core when detaching the battery assembly, and prevents a shortcoming that the atomization core is unplugged by the user when rotating the battery sleeve. Secondly, a plug-in groove configured for accommodating the second electric connection assembly is defined on the first electric connection assembly, and the second electric connection assembly is inserted into the plug-in groove to increase contact area between electrodes of the first electric connection assembly and the second electric connection assembly. Thirdly, the atomization core and the e-liquid cup assembly are in detachable connection, which is convenient for users to replace the damaged atomization core to prevent waste of resource. Finally, the e-liquid storage sleeve is made of light permeable materials, which is convenient for users to check residual e-liquid in the e-liquid storage cavity.

### BRIEF DESCRIPTION OF THE DRAWINGS

The present invention will be further described with reference to the accompanying drawings and embodiments in the following, in the accompanying drawings:

FIG. 1 is a structure schematic diagram of an electronic cigarette provided by a first preferable embodiment of present invention;

FIG. 2 is a structure schematic diagram of a battery assembly of the electronic cigarette of FIG. 1;

FIG. 3 is a structure schematic diagram of an e-liquid cup assembly and a suction nozzle of the electronic cigarette of FIG. 1;

FIG. 4 is a structure schematic diagram of an atomization core of the electronic cigarette of FIG. 1;

FIG. 5 is a schematic diagram of an atomization sleeve of the atomization core of FIG. 4;

FIG. 6 is block diagram of the atomization core of FIG. 4;

FIG. 7 is a structure schematic diagram of an electronic cigarette provided by a second preferable embodiment of present invention;

FIG. 8 is a structure schematic diagram of the atomization assembly of FIG. 7;

FIG. 9 is a structure schematic diagram of an electronic cigarette provided by a third preferable embodiment of present invention.

### DETAILED DESCRIPTION OF THE PREFERRED EMBODIMENT

To make the technical feature, objective and effect of the present application be understood more clearly, now the specific implementation of the present application is described in detail with reference to the accompanying drawings and embodiments.

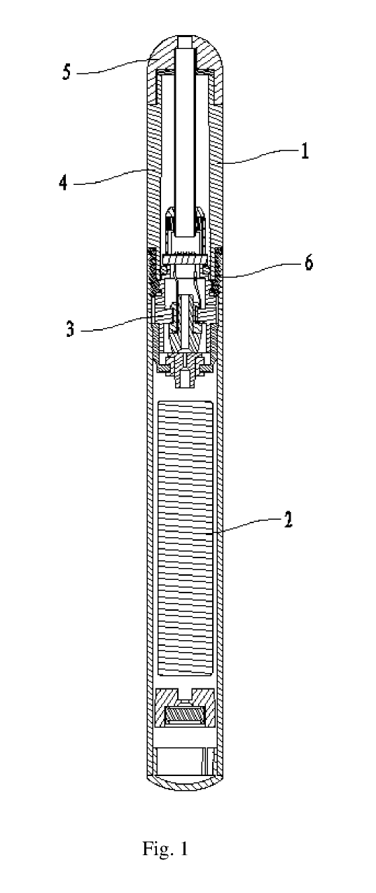

FIG. 1 shows an electronic cigarette provided by one of the preferable embodiment of present invention, the electronic cigarette comprises an atomization assembly 1 and a battery assembly 2, the battery assembly 2 is electrically connected to the atomization assembly 1 and is configured for supplying electrical power to the atomization assembly 1; the atomization assembly 1 comprises an e-liquid cup assembly 4, an atomization core 3 and a suction nozzle 5, the atomization core 3 and the suction nozzle 5 are respectively connected to two ends of the e-liquid cup assembly 4.

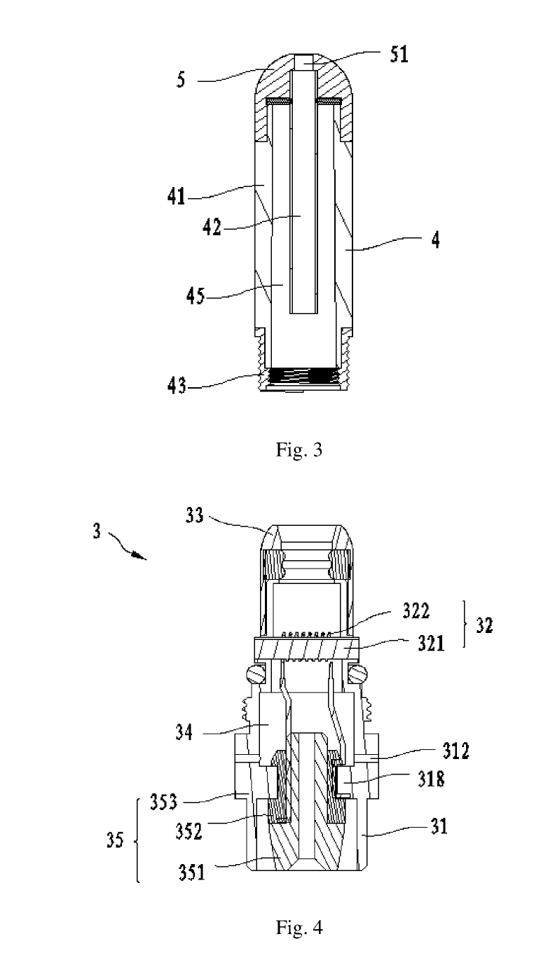

Specifically, with reference to FIG. 3, the e-liquid cup assembly 4 comprises a light permeable e-liquid storage sleeve 41, an e-liquid storage cavity 45 configured for storing e-liquid is formed in the e-liquid storage sleeve 41, an end of the e-liquid storage sleeve 41 is an open end in communication with the e-liquid storage cavity 45, a connector 43 is provided at the open end, an atomization core 3 detachably connected to the connector 43 and configured for atomizing the e-liquid is inserted in the connector 43.

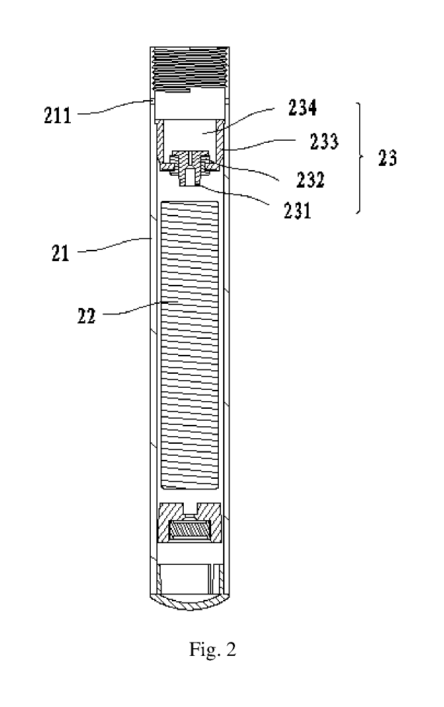

With reference to FIG. 2, the battery assembly 2 comprises a battery sleeve 21, a connecting structure 6 configured for detachably connecting the battery sleeve 21 with the e-liquid storage sleeve 41 is provided at a connection of the battery sleeve 21 and the e-liquid storage sleeve 41 (with reference to FIG. 1), a first electric connection assembly 23 electrically connected to the atomization core 3 is accommodated in an end of the battery sleeve 21.

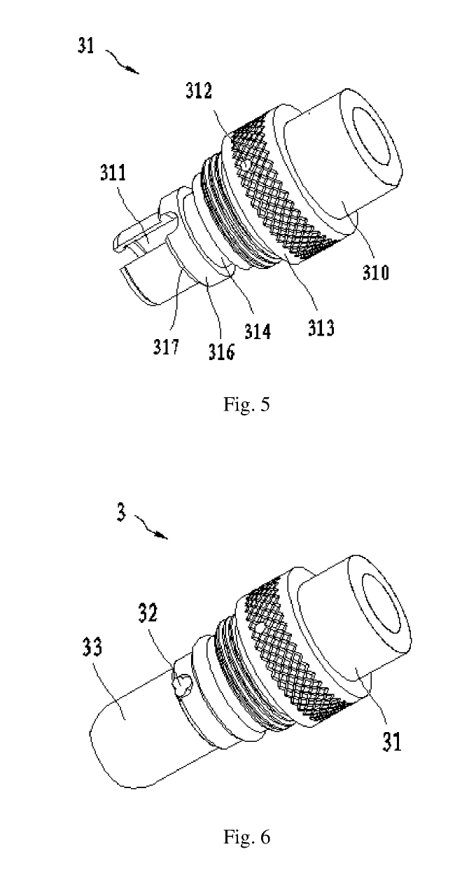

With reference to FIG. 4, the atomization core 3 comprises an atomization sleeve 31, a second electric connection assembly 35 electrically connected to the first electric connection assembly 23 is provided at an end of the atomization sleeve 31 which extends outside the e-liquid storage sleeve 41, the second electric connection assembly 35 is inserted into the battery sleeve 21 and is abutted against the first electric connection assembly 23, an atomization cavity 34 is formed in the atomization sleeve 31, a heating wire assembly 32 electrically connected to the second electric connection assembly 35 is accommodated in the atomization cavity 34.

In the electronic cigarette of the prior art, the atomization core 3 is detachably connected to the e-liquid storage sleeve 41, while the battery assembly 2 is detachably connected to the atomization core 3. When an user disassembles the battery assembly 2 from an end of the atomization core 3, if connection strength between the atomization core 3 and the e-liquid storage sleeve 41 is smaller than connection strength between the atomization core 3 and the battery assembly 2, the user easily rotates the atomization core 3 out from the e-liquid storage sleeve 41, while the atomization core 3 and the battery assembly 2 are still connected with each other, which causes problems such as a leakage of e-liquid and affects experience of the user.

In this embodiment, the battery sleeve 21 is directly detachably connected to the e-liquid storage sleeve 41, which prevents the user from applying a force directly to the atomization core 3 when detaching the battery assembly 2, and prevents a shortcoming that the atomization core 3 is unplugged by the user when rotating the battery sleeve 21.

As shown in FIG. 1, the battery sleeve 21 is detachably connected to the e-liquid storage sleeve 41 through the connecting structure 6, the connecting structure 6 is a threaded connecting structure. Specifically, the connecting structure 6 comprises an external thread defined on an outer wall of the connector 43 (not shown in the figures), and an internal thread (not shown in the figures) matched with the external thread and defined on the battery sleeve 21.

Further, with reference to FIG. 2 and FIG. 4, in this embodiment, the second electric connection assembly 35 of the atomization core 3 is inserted into the battery sleeve 21 and abuts against the first electric connection assembly 23, so as to increase contact area between the first electric connection assembly 23 and the second electric connection assembly 35, thus to make the electrical connection between the battery assembly 2 and the atomization core 3 be more stable.

Further, at least one inlet hole communicating with the atomization cavity 34 is defined on the battery sleeve 21 and the atomization sleeve 31, the inlet hole is configured for inletting air and discharging the e-liquid in the atomization cavity 34. As the inlet hole is located outside the e-liquid storage sleeve 41, thus noise will not be produced when airflow goes into the atomization cavity 34 through the inlet hole. With reference to FIG. 2 and FIG. 4, the inlet hole comprises a first inlet hole 211 defined on the battery sleeve 21 and a second inlet hole 312 defined on the atomization sleeve 31, the second inlet hole 312 is respectively in communication with the atomization cavity 34 and the first inlet hole 211.

Preferably, the second inlet hole 312 is located at a wall of the atomization cavity 34 close to the battery assembly 2 and is configured for discharging the e-liquid at the wall of atomization cavity 34.

A specific structure of the battery assembly 2 is specifically described with reference to the embodiments shown in FIG. 1 and FIG. 2.

As shown in FIG. 2, the battery assembly 2 mainly comprises a battery sleeve 21, a battery 22, a first electric connection assembly 23, an airflow sensor and a control unit (not labeled in the figures).

The battery sleeve 21 is roughly a hollow cylinder structure, at least one first inlet hole 211 communicating with the atomization cavity 34 is defined on an outer wall of the battery sleeve.

The battery 22 is fixed in the battery sleeve 21 and is mainly configured for supplying required power for the atomization core 3 and the control unit.

The first electric connection assembly 23 is fixed in an end of the battery sleeve 21 which is configured for plug-in connecting to the atomization core 3, and the first electric connection assembly 23 is electrically connected to the battery 22 and is configured for electrically connected to the second electric connection assembly 35 of the atomization core 3.

In this embodiment, the first electric connection assembly 23 comprises a first internal electrode 231, a first insulation member 232 and a first external electrode 233 which are successively sheathed and connected from inside to outside, the first external electrode 233 is fixed on an inner wall of the battery sleeve 21. Specifically, an annular convex plate (not labeled in the figures) is bulged on a lateral margin of an end of the first external electrode towards the battery assembly 2, an annular groove (not labeled in the figures) fitting with the annular convex plate is buckled on the inner wall of the battery sleeve 21, the first external electrode 233 is fixed on the inner wall of the battery sleeve 21 through the annular convex plate.

With reference to FIG. 4, the second electric connection assembly 35 comprises a second internal electrode 351, a second insulation member 352 and a second external electrode 353 which are successively sheathed and connected from inside to outside. The first internal electrode 231 is electrically connected to the second internal electrode 351, the first external electrode 233 is electrically connected to the second external electrode 353.

In this embodiment, the second external electrode 353 is the atomization sleeve 31 of the atomization core 3, understandably, in other embodiments, the second external electrode 353 can be not the atomization sleeve 31, but the second external electrode 353 is fixed in the atomization sleeve 31. The present invention not limits specific structures of the first electric connection assembly 23 and the second electric connection assembly 35, as long as the battery assembly 2 is enabled to be electrically connected to the atomization core 3.

Preferably, a plug-in groove 234 (as shown in FIG. 2) configured for accommodating the second electric connection assembly 35 is defined on the first external electrode 233, the second electric connection assembly 35 is inserted into the plug-in groove 234, so as to make the atomization sleeve 31 abut against a wall of the plug-in groove 234, the second internal electrode 351 abuts against an end surface of the first internal electrode 231 to increase contact area of electrodes of the first electric connection assembly 23 and the second electric connection assembly 35.

The airflow sensor and the control unit are respectively accommodated in the battery sleeve 21, the airflow sensor is mainly configured for detecting an airflow signal and producing a pulse signal, the control unit is respectively electrically connected to the airflow sensor and the battery 22. The control unit is mainly configured for receiving a signal transmitted by the airflow sensor and controlling a working state of the atomization core 3 according to the received signal. Certainly, the airflow sensor and the control unit can be replaced by a key switch to control whether the battery 22 supplies the electrical power to the atomization core 3, it is not limited here.

In this embodiment, the battery assembly 2 comprises the battery 22, the first electric connection assembly 23, the airflow sensor and the control unit which are respectively sheathed in the battery sleeve 21, understandably, the structure of the battery assembly 2 of present invention is not limited above and can be other structures, as long as the battery assembly 2 can supply power for the atomization assembly 1 and the battery sleeve 21 is detachably connected to the e-liquid storage sleeve 41.

Specific structures of the e-liquid cup assembly 4 and a suction nozzle 5 of the atomization assembly 1 are specifically described with reference to the embodiments shown in FIG. 1 and FIG. 3.

As shown in FIG. 3, the e-liquid cup assembly 4 mainly comprises the e-liquid storage sleeve 41, a vent pipe 42, a connector 43 and the e-liquid storage cavity 45.

The e-liquid storage sleeve 41 is roughly a cylinder structure, the connector 43 and the suction nozzle 5 are respectively connected to two ends of the e-liquid storage sleeve 41, the vent pipe 42 is defined in the e-liquid storage sleeve 41 along an axial of the e-liquid storage sleeve 41, an e-liquid storage cavity 45 is formed between the e-liquid storage sleeve 41 and the vent pipe 42, for the atomization core 3, e-liquid configured for being atomized is stored in the e-liquid storage cavity 45. In order to make it convenient for users to check residual e-liquid in the e-liquid storage cavity 45, in this embodiment, the e-liquid storage sleeve 41 is made of light permeable materials.

Two ends of the vent pipe 42 are respectively inserted into the suction nozzle 5 and the atomization core 3, an airflow channel configured for air flowing to the suction nozzle 5 is enclosed in the vent pipe 42, and the e-liquid storage cavity 45 configured for storing e-liquid is formed between the vent pipe 42 and the e-liquid storage sleeve 41.

The connector 43 is inserted in an end of the e-liquid storage sleeve 41 configured for connecting to the atomization core 3, specifically, an outer circumference surface of the connector 43 is detachably connected to the battery sleeve 21, an inner wall of the connector 43 is detachably connected to the atomization core 3. In this embodiment, the inner wall of the connector 43 and the atomization core 3 are connected through screw threads, an internal thread is defined at a position of the connector 43 configured for connecting to the atomization core 3, an external thread structure fitting with the internal thread is defined on an outer circumference surface of the atomization core 3. If other detachable connection structures are provided between the connector 43 and the atomization core 3, other connection structures can be arranged on the connector 43, such as an elastic buckling shrapnel of a buckling connection, this embodiment merely takes a threaded connection as an example but not limits the connection as screw threads connection.

As shown in FIG. 3, the suction nozzle 5 is fixed on an end of the e-liquid storage sleeve 41 opposite to the atomization core 3, two ends of the vent pipe 42 are respectively inserted into the suction nozzle 5 and the atomization core 3, an outlet hole 51 communicating with the vent pipe 42 is defined in the suction nozzle 5, smoke atomized and produced by the atomization core 3 successively runs through the vent pipe 42 and the outlet hole 51 then is sucked by the user.

Preferably, an external end surface of the suction nozzle 5 is a spherical surface, to make the suction nozzle 5 be more artistic, and the suction nozzle 5 with the spherical surface as the external end surface is more convenient for cleaning dirt. Besides, the structure of the suction nozzle 5 with the spherical surface as the external end surface has good stability, so as to prevent the suction nozzle 5 from being transformed and falling off when hard objects such as teeth of mouth clamps on the suction nozzle 5.

Preferably, the suction nozzle 5 of this embodiment is made of metal materials to prevent the suction nozzle 5 from being transformed and falling off when hard objects such as teeth of mouth clamping on the suction nozzle 5. The metal can be copper, iron or steel, it is not limited here.

Specific structures of the atomization core 3 of the atomization assembly 1 are specifically described with reference to the embodiments shown in FIG. 4, FIG. 5 and FIG. 6.

As shown in FIG. 4, the atomization core 3 mainly comprises the atomization sleeve 3, the heating wire assembly 32, the atomization cover 33, the atomization cavity 34 and the second electric connection assembly 35.

The atomization cavity 34 is defined inside the atomization sleeve 31 and is communicated with the vent pipe 42, heating wire assembly 32 is accommodated in the atomization cavity 34; the atomization cover 33 is sleeved on the atomization sleeve 31 and the vent pipe 42, and abuts against the heating wire assembly 32, an end of the vent pipe 42 goes through the atomization cover 33 and extends into the atomization cavity 34.

As shown in FIG. 5, one end of the atomization sleeve 31 is detachably connected to the e-liquid storage sleeve 41, the other end extends into the battery sleeve 21 and is in plug-in connection with the battery sleeve 21. Specifically, the atomization sleeve 31 comprises a main body 310 which is roughly a hollow cylinder structure, an installation groove 311, the second inlet 312, a limiting buckle 313, an annular groove 314, a convex 316, a limiting convex part 317 and an annular buckle 318 are defined on the main body 310 (with reference to FIG. 5).

The installation groove 311 is defined on an end of the main body 310 close to the suction nozzle 5, and extends towards the side opposite to the suction nozzle 5. In this embodiment, there are two installation grooves 311, two installation grooves 311 separated with each other and in communication with the e-liquid storage cavity 45 are defined relatively and axially on an end of a sidewall of the atomization sleeve 31 opposite to the battery assembly 2, the heating wire assembly 32 is built in the bottom of the two installation grooves 311.

The second inlet hole 312 is defined on an end extending outside e-liquid storage sleeve 41 of the main body 310 and is respectively communicated with the atomization cavity 34 and the first inlet hole 211 which is on the battery sleeve 21, the second inlet hole 312 is mainly configured for air inflowing and discharging the e-liquid in the atomization cavity 34. As the second inlet hole 312 is located outside the e-liquid storage sleeve 41, thus airflow directly flows into the atomization cavity 34 through the second inlet hole 312 without producing noise.

The limiting buckle 313 is bulged on an outer wall of the main body 310 to prevent the connector 43 from blocking the inlet hole 312 during an assembling process. Specifically, during the assembling process of the atomization core 3, the limiting buckle 313 abuts against a lateral margin of an end of the connector 43, the inlet hole 312 is located at a side of the limiting buckle 313 opposite to the suction nozzle 5 to prevent the inlet hole 312 from being blocked during the assembling process.

Preferably, when the atomization core 3 is needed to be taken out, in order to make it be convenient for the user to rotate the atomization sleeve 31, a knurl structure can be defined on the atomization sleeve 31 (not labeled in the figure).

Preferably, in order to prevent the e-liquid leaking from the connection between the main body 310 and the e-liquid storage sleeve 41, a sealing element (not labeled in the figure) can be sleeved on the main body 310. Specifically, in this embodiment, a clamping groove 314 is defined on the outer circumference wall of the main body 310 located at the e-liquid storage sleeve 41, the sealing element is fixed in the clamping groove 314 and elastically abuts against the inner wall of the e-liquid storage sleeve 41 to realize the function of sealing.

Preferably, in order to better support the heating wire assembly 32, the convex 316 configured for supporting the heating wire assembly 32 is defined on the main body 310, one end of the convex 316 is fixed on the bottom of the installation groove 311, the other end extends towards the direction opposite to an axis of the main body 310. The arrangement of the convex 316 can prevent the heating wire assembly 32 falling into the atomization cavity 34 as the side wall supporting the heating wire assembly 32 is too thin.

Preferably, with reference to FIG. 6, as the atomization cover 33 is sheathed on the atomization sleeve 31 and abuts against the heating wire assembly 32, in order to prevent the atomization cover 33 from excessively extruding the heating wire assembly 32, the limiting convex part 317 abutting against the end surface of the atomization cover 33 can be bulged on the main body 310.

As shown in FIG. 4, the heating wire assembly 32 comprises an e-liquid guide member 321 and a heating wire 322, the e-liquid guide member 321 is made of oil absorption materials and is fixed on the bottom of the installation groove 311, and two ends of the e-liquid guide member 321 respectively extends into the e-liquid storage cavity 45, the heating wire 322 is surrounded on the e-liquid guide member 321 and is electrically connected to the battery assembly 2. As the heating wire assembly 32 is a common method in the prior art, thus the specific structure is not described anymore.

As shown in FIG. 4 and with reference to FIG. 5 and FIG. 6, an accommodation cavity (not labeled in the figures) is defined throughout the atomization cover 33 axially, an end of the atomization cover 33 opposite to the suction nozzle 5 is sheathed on the main body 310 and abuts against the limiting convex part 317, the vent pipe 42 (with reference to FIG. 3) inserts from an end of the atomization cover 33 close to the suction nozzle 5 and is in an interference fit with a sidewall of the accommodation cavity.

Preferably, to prevent the e-liquid leaking from the connection between the vent pipe 42 and the atomization cover 33, a sealing ring (not labeled in the figures) can be defined in the accommodation cavity of the atomization cover 33 to sleeve on the vent pipe 42, an outer circumference surface of the sealing ring elastically abuts against the sidewall of the accommodation cavity, an inner circumference surface of the sealing ring elastically abuts against an outer circumference surface of the vent pipe 42 to play a sealing role.

As shown in FIG. 4, the second electric connection assembly 35 is positioned at an end of the atomization sleeve 31 close to the battery assembly 2 and is configured for electrically connected to the battery assembly 2 and the heating wire assembly 32. In this embodiment, the second electric connection assembly 35 is fixed on the annular buckle 318 in the atomization sleeve 3. The annular buckle 318 is defined at an end of the atomization sleeve 31 configured for connecting to the battery assembly 2, and one end of the annular buckle 318 is fixed on the sidewall of the atomization cavity 34, the other end extends towards an axial close to the atomization sleeve 31 and forms an center hole (not labeled in the figure), the second electric connection assembly 35 is fixed at the center hole.

In this embodiment, the atomization core 3 mainly comprises the atomization sleeve 31, the atomization cavity 34 is defined in the atomization sleeve 31 and is communicated with the vent pipe 42, the heating wire assembly 32 is accommodated in the atomization cavity 34; the atomization cover 33 is sheathed on the atomization sleeve 31 and the vent pipe 42 and abuts against the heating wire assembly 32, an end of the vent pipe 42 runs through the atomization cover 33 and extends into the atomization cavity 34. Understandably, the structure of the atomization core 3 of present invention is not limited above and can be other structures, as long as it can atomize the e-liquid.

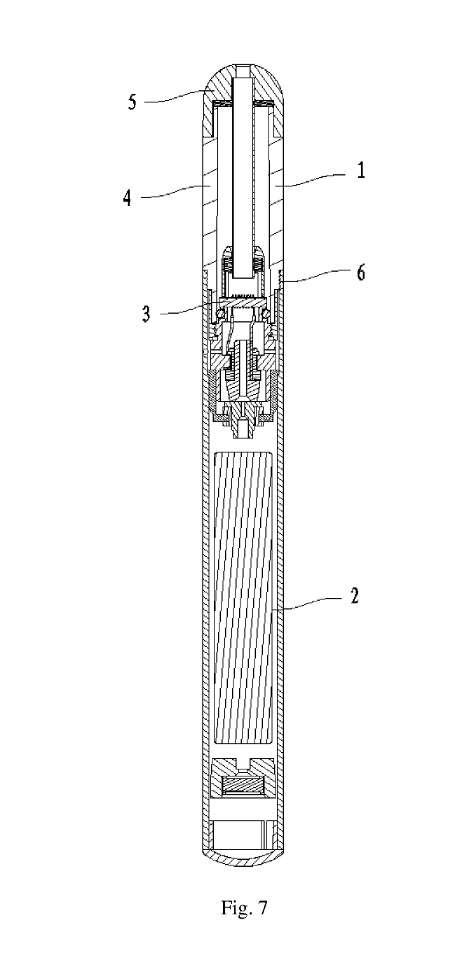

FIG. 7 shows an electronic cigarette provided by a second preferable embodiment of present invention, a difference from the first embodiment lies in the position of the external thread on the connecting structure 6.

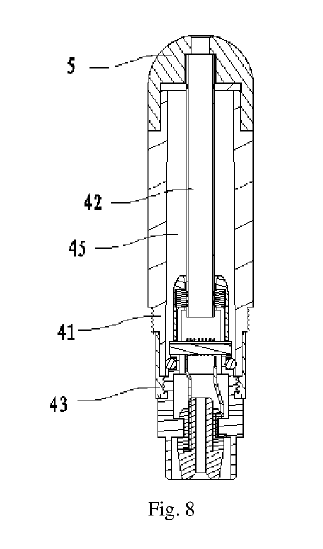

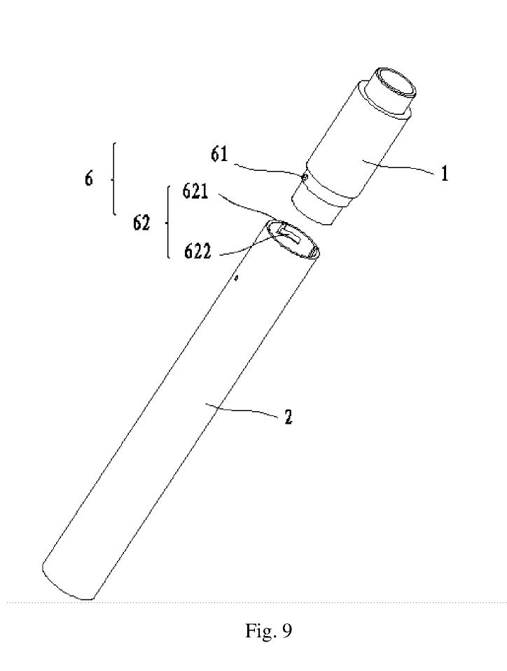

As shown in FIG. 8, in this embodiment, the connecting structure 6 is a threaded connecting structure, but the connecting structure 6 comprises an external thread (not labeled in the figure) defined on the e-liquid storage sleeve 41 and an internal thread (not labeled in the figure) fitting with the external thread and defined on the battery sleeve 21.

FIG. 9 shows an electronic cigarette provided by a third preferable embodiment of present invention, a difference from the first embodiment lies in the structure of the connecting structure 6.

As shown in FIG. 9, in this embodiment, the connecting structure 6 is bayonet connecting structure, the connecting structure 6 comprises a buckle 61 and a clamping groove 61 which are buckled with each other. The buckle 61 is bulged on an outer wall of the e-liquid storage sleeve 41, the clamping groove 62 is defined on an inner wall of the battery sleeve 21.

Specifically, the clamping groove 62 is shaped in “L”, the clamping groove 62 comprises an insertion channel 621 and a rotation channel 622, the insertion channel 621 and the rotation channel 622 are smoothly connected to each other; the insertion channel 621 is defined on an end of the battery sleeve 21 and extends along an axis of the battery sleeve 21 to a side opposite to the buckle 61, the rotation channel 622 is connected to an end of the insertion channel 621 opposite to the buckle 61 and extends along a circumference of the battery sleeve 21.

During an assembling, firstly, moving the buckle 61 on the e-liquid storage sleeve 41 along the insertion channel 621 to the bottom of the insertion channel 621, then rotating the atomization assembly 1 to make the buckle 61 move into the rotation channel 622, so as to realize a detachable connection between a battery assembly 2 and an atomization assembly 1.

In this embodiment, the number of the buckle 61 and the clamping groove 62 is four, respectively. Understandably, the specific number of the buckle 61 and the clamping groove 62 are not limited in present invention, as long as the bayonet connection between the battery sleeve 21 and the e-liquid storage sleeve 41 can be realized.

To sum up, the implementation of the electronic cigarette of present invention has following beneficial effects:

(1) The battery sleeve is directly detachably connected to the e-liquid storage sleeve, which prevents a user from applying a force directly to the atomization core when detaching the battery assembly, and prevents the shortcoming that the atomization core is unplugged by the user when rotating the battery sleeve.

(2) A plug-in groove configured for accommodating the second electric connection assembly is defined on the first electric connection assembly, and the second electric connection assembly is inserted into the plug-in groove to increase the contact area between the electrodes of the first electric connection assembly and the second electric connection assembly.

(3) The atomization core and the e-liquid cup assembly are in detachable connection, which is convenient for users to replace the damaged atomization core to prevent waste of resource.

(4) The e-liquid storage sleeve is made of light permeable materials, which is convenient for users to check residual e-liquid in the e-liquid storage cavity.

While the present invention has been described with reference to preferred embodiments above, however the present invention is not limited to above-mentioned embodiments, those modifications, improvements and equivalent substitutions, which do not depart from the scope of the spirit and the principle of the present invention, should be included within the scope of the present invention.

1. An electronic cigarette comprising an atomization assembly (1) and a battery assembly (2), the battery assembly (2) is electrically connected to the atomization assembly (1) and is configured for supplying electrical power to the atomization assembly (1), wherein the atomization assembly (1) comprises a light permeable e-liquid storage sleeve (41), an e-liquid storage cavity (45) configured for storing e-liquid is formed in the e-liquid storage sleeve (41), an end of the e-liquid storage sleeve (41) is an open end in communication with the e-liquid storage cavity (45), a connector (43) is provided at the open end, an atomization core (3) detachably connected to the connector (43) and configured for atomizing the e-liquid is inserted in the connector (43);
the battery assembly (2) comprises a battery sleeve (21), a connecting structure (6) configured for detachably connecting the battery sleeve (21) to the e-liquid storage sleeve (41) is provided at a connection of the battery sleeve (21) and the e-liquid storage sleeve (41), a first electric connection assembly (23) electrically connected to the atomization core (3) is accommodated in an end of the battery sleeve (21);
the atomization core (3) comprises an atomization sleeve (31), a second electric connection assembly (35) electrically connected to the first electric connection assembly (23) is provided at an end of the atomization sleeve (31), the end of the atomization sleeve (31) extends outside the e-liquid storage sleeve (41), the second electric connection assembly (35) is inserted into the battery sleeve (21) and is abutted against the first electric connection assembly (23), an atomization cavity (34) is formed in the atomization sleeve (31), a heating wire assembly (32) electrically connected to the second electric connection assembly (35) is accommodated in the atomization cavity (34).

2. The electronic cigarette according to claim 1, wherein the connecting structure (6) is a threaded connecting structure or a bayonet connecting structure.

3. The electronic cigarette according to claim 2, wherein the connecting structure (6) comprises an external thread defined on an outer wall of the connector (43) or the e-liquid storage sleeve (41), and an internal thread matched with the external thread and defined on the battery sleeve (21).

4. The electronic cigarette according to claim 2, wherein the connecting structure (6) comprises at least one buckle (61) bulging on an outer wall of the e-liquid storage sleeve (41), and at least one clamping groove (62) defined on an inner wall of the battery sleeve (21), the clamping groove (62) is shaped in “L” and matched with the buckle (61).

5. The electronic cigarette according to claim 4, wherein the clamping groove (62) comprises an insertion channel (621) and a rotation channel (622), the insertion channel (621) and the rotation channel (622) are smoothly connected to each other; the insertion channel (621) is defined on an end of the battery sleeve (21) and extends along an axis of the battery sleeve (21) to a side opposite to the buckle (61), the rotation channel (622) is connected to an end of the insertion channel (621) opposite to the buckle (61) and extends along a circumference of the battery sleeve (21).

6. The electronic cigarette according to claim 1, wherein at least one inlet hole communicating with the atomization cavity (34) and configured for inletting air and discharging the e-liquid in the atomization cavity (34) is defined on an outer wall of the battery sleeve (21) and the atomization sleeve (31), the inlet hole comprises a first inlet hole (211) defined on the battery sleeve (21) and a second inlet hole (312) defined on the atomization sleeve (31).

7. The electronic cigarette according to claim 6, wherein the second inlet hole (312) is located at a wall of an end of the atomization cavity (34) and is configured for discharging the e-liquid at the wall of atomization cavity (34), the end of the atomization cavity (34) is close to the battery assembly (2).

8. The electronic cigarette according to claim 1, wherein the first electric connection assembly (23) is fixed on an inner wall of the battery sleeve (21), a plug-in groove (234) plug-in connected to the second electric connection assembly (35) is defined on a side of the first electric connection assembly (23) towards the atomization core (3).

9. The electronic cigarette according to claim 8, wherein the first electric connection assembly (23) comprises a first internal electrode (231), a first insulation member (232) and a first external electrode (233) which are successively sheathed and connected from inside to outside, the plug-in groove (234) is defined on the first external electrode (233).

10. The electronic cigarette according to claim 1, wherein two installation grooves (311) separated with each other and in communication with the e-liquid storage cavity (45) are defined relatively and axially on an end of a sidewall of the atomization sleeve (31), the end of the sidewall of the atomization sleeve (31) is opposite to the battery assembly (2), the heating wire assembly (32) is arranged in the two installation grooves (311).

11. The electronic cigarette according to claim 10, wherein the heating wire assembly (32) comprises an e-liquid guide member (321) configured for absorbing the e-liquid in the e-liquid storage cavity (45), and a heating wire (322) configured for atomizing the e-liquid on the e-liquid guide member (321), two ends of the heating wire (322) are respectively and electrically connected to the second electric connection assembly (35) and realize an electrical connection with the battery assembly (2) through the second electric connection assembly (35).

12. The electronic cigarette according to claim 1, wherein a suction nozzle (5) is connected to an end of the e-liquid storage sleeve (41), the end of the e-liquid storage sleeve (41) is opposite to the atomization core (3), a vent pipe (42) is sheathed in the e-liquid storage sleeve (41), two ends of the vent pipe (42) are respectively inserted into the suction nozzle (5) and the atomization core (3).

13. The electronic cigarette according to claim 12, wherein an external end surface of the suction nozzle (5) is a spherical surface.

14. The electronic cigarette according to claim 12, wherein the atomization core (3) further comprises an atomization cover (33) sheathed on the atomization sleeve (31) and the vent pipe (42) and abutted against the heating wire assembly (32), an end of the vent pipe (42) runs through the atomization cover (33) and extends into the atomization cavity (34).

15. The electronic cigarette according to claim 14, wherein a limiting convex part (317) abutting against an end surface of the atomization cover (33) is defined on an outside surface of the atomization sleeve (31).

16. The electronic cigarette according to claim 14, wherein a convex (316) configured for supporting the heating wire assembly (32) is defined on an outside surface of the atomization sleeve (31).

17. An electronic cigarette comprising an atomization assembly (1) and a battery assembly (2), the battery assembly (2) is electrically connected to the atomization assembly (1) and is configured for supplying electrical power to the atomization assembly (1), wherein the atomization assembly (1) comprises a light permeable e-liquid storage sleeve (41), an e-liquid storage cavity (45) configured for storing e-liquid is formed in the e-liquid storage sleeve (41), an end of the e-liquid storage sleeve (41) is an open end in communication with the e-liquid storage cavity (45), a connector (43) is provided at the open end, an atomization core (3) detachably connected to the connector (43) and configured for atomizing the e-liquid is inserted in the connector (43);
the battery assembly (2) comprises a battery sleeve (21), the battery sleeve (21) is detachably connected to the e-liquid storage sleeve (41) through a threaded connecting structure or a bayonet connecting structure, a first electric connection assembly (23) electrically connected to the atomization core (3) is accommodated in an end of the battery sleeve (21);
the atomization core (3) comprises an atomization sleeve (31), a second electric connection assembly (35) electrically connected to the first electric connection assembly (23) is provided at an end of the atomization sleeve (31), the end of the atomization sleeve (31) extends outside the e-liquid storage sleeve (41), the second electric connection assembly (35) is inserted into the battery sleeve (21) and is abutted against the first electric connection assembly (23), an atomization cavity (34) is formed in the atomization sleeve (31), a heating wire assembly (32) electrically connected to the second electric connection assembly (35) is accommodated in the atomization cavity (34).
